(19) United States
(12) Patent Application Publication
(10) Pub. No.: US 2011/0295358 A1
(43) Pub. Date: Dec. 1, 2011

(54) ENDOLUMINAL PROSTHESIS

(76) Inventors: Silvio Rudolph Schaffner, Berlingen (CH); Gianpiero Spirito, Pietra Ligure (IT)

(21) Appl. No.: 13/140,413

(22) PCT Filed: Dec. 29, 2008
(86) PCT No.: PCT/IT2008/000803

§ 371 Date: Aug. 18, 2011

(51) Int. Cl. A61F 2/82

(52) U.S. Cl. 623/1.15

(57) ABSTRACT

An endoluminal prosthesis (500) comprises a tubular body (502) suitable to turn from a collapsed state to an expaneded or partially expanded state. The tubular body develops along a longitudinal axis (504) and comprises a plurality of closed paths (506a, 506b, 506c, 506d) developing along a substantially circumferential direction. The paths are serially arranged along the longitudinal axis. Each of said paths is connected to the one(s) immediately adjacent. Moreover, each of said paths comprises strut portions, or struts (508), each of which is characterized in that the ratio of the strut length (L) to the strut width (W) is of at least 20.

The present invention relates to an endoluminal prosthesis suitable for implantation in passageways or conduits of the human or animal body, such as blood vessels.

Endoluminal prosthesis are known in the prior art.

WO 03/030786 discloses a stent in which the interconnection strut width is varied along the strut length in order to suitably distribute tensile strain during radial expansion and contraction of the stent. This document also discloses a specific relation between length and width at the interconnection of two consecutive struts of the same serpentine.

U.S. Pat. No. 6,862,124 discloses a stent in which the struts at the stent ends have a mass greater that the mass of the struts at the stent central region, the different mass being obtained, for instance, by acting on the strut length and width.

US 2006/0224231 discloses a stent in which the connection between two consecutive struts of the same serpentine has a width greater than the strut width.

US 2008/0221661 discloses a stent having struts of the same serpentine with different widths.

WO 2005/120394 discloses a stent with struts having different widths along the same strut length.

EP 0 886 501 discloses an expandable stent in which there is substantially no lengthening or shortening of the stent between radially compressed or expanded states. The stent comprises a flexible, tubular-shaped body which is formed by interconnected, closed lattice cells, said cells being formed from a plurality of filaments bent to form the lattice cells and wound about one another at locations where the cell filaments meet so as to fix the lattice cells to one another in longitudinal and circumferential directions.

In particular, the present invention relates to an endoluminal prosthesis suitable for implantation at the site of an aneurysm, thereby preventing or slowing down the growth of the aneurysm and reducing the probability of aneurysm rupture.

An aneurysm is a localized, blood-filled dilation (that is to say, a balloon-like bulge) of a blood vessel caused by disease or weakening of the vessel wall. Aneurysms most commonly occur in arteries at the base of the brain (the so-called “Circle of Willis”) or in the aorta. The bulge in the blood vessel can burst and lead to death at any time. The larger an aneurysm becomes, the more likely it is to burst and, since an aneurysm naturally grows, it will inevitably reach the bursting point if it remains undetected.

In case an aneurysm of a body vessel occurs, two main options are currently available to the surgeon.

The first option would be to carry out a surgical intervention. Typically, the surgical intervention consists in clipping the artery, e.g. closing the base of the aneurysm with a clip or, especially in the case of aortic aneurysm, reconstructing the artery with a tube graft, an aortic iliac bifurcation graft or an aortofemoral bypass. This kind of procedure is quite invasive and involves very high risks for the patient's life as well as a long recovery period after the intervention.

The second option is to perform an endovascular procedure. When a suitable size of the aneurysm is reached, the surgeon treats the damaged area by implanting an endoluminal prosthesis to exclude the sac from circulation.

Since aneurysms are typically detected at a very early stage, the patient could have to wait for a few years in some instances before the aneurysm reaches a suitable size for the surgical or endovascular procedures, also taking into account that typically the aneurysm growth rate is of about 1-2 mm per year.

Nowadays the endovascular procedure is generally carried out by using a covered stent, that is to say a stent provided with an outer impermeable sleeve, so that the blood flow is prevented from acting directly onto the vessel wall thereby causing the aneurysm to grow further.

Even though the endovascular procedure is less invasive and can be considered a good option in high risk patients, such procedure still suffers from serious drawbacks. In particular, some time after the stent implantation, leakages can occur between the covered stent and the vessel wall. This aspect is greatly undesirable since the presence of blood flowing along the vessel wall and radially external to the covered stent contributes to the detachment of the covered stent from the damaged area, which causes the aneurysm to begin growing again. Leakage can be due to incompetent sealing zones (generally known as “Type 1”), to back flow from collateral vessels feeding (“Type 2”), to incomplete connection at junctions (“Type 3”) or finally to structural defects (“Type 4”). These complications usually require second challenging interventions or open conversion.

Furthermore, a correct and stable position of the covered stent in correspondence of the damaged area cannot be ensured. In fact, it has been observed that the covered stent can move from the treated area, thereby making the implant procedure fruitless. The covered stent can move since it is not enough anchored to the vessel wall. In order to increase anchorage of the covered stent to the vessel wall, anchoring or gripping means, such as pins, can be provided at some suitable locations, for example at the ends of the stent. However, said anchoring or gripping means operate as hooks acting on the vessel wall and the latter can be damaged, even seriously damaged, by said means which puncture the vessel wall.

The Applicant has perceived the need of providing an endoluminal prosthesis suitable for implantation at the damaged area of a blood vessel, that is to say the area where an aneurysm has occurred, which can advantageously and suitably support the vessel wall while at the same time avoiding the latter to be deformed by the blood flowing within the vessel thereby causing the aneurysm to grow further.

A further need is to provide an endoluminal prosthesis which does not include anchoring or gripping means capable of damaging the vessel wall but that, once delivered to the site of the aneurysm to be treated, is not easily displaced.

In order to meet these and other needs and to overcome the drawbacks of the prior art, the present invention provides an endoluminal prosthesis comprising:

a tubular body suitable to turn from a collapsed state to an expanded or partially expanded state,

said tubular body developing along a longitudinal axis,

said tubular body comprising a plurality of closed paths developing along a substantially circumferential direction,

said paths being serially arranged along the longitudinal axis,

each of said paths being connected to the one(s) immediately adjacent,

each of said paths comprising strut portions, or struts,

each strut portion being characterized in that the ratio of the strut length to the strut width is of at least 20.

Further embodiments of the endoluminal prosthesis of the invention are described by the dependent claims.

Further features and advantages of the endoluminal prosthesis of the invention will become apparent from the description provided below of preferred embodiments thereof, being merely illustrative and non-limiting, with reference to the annexed drawings in which:

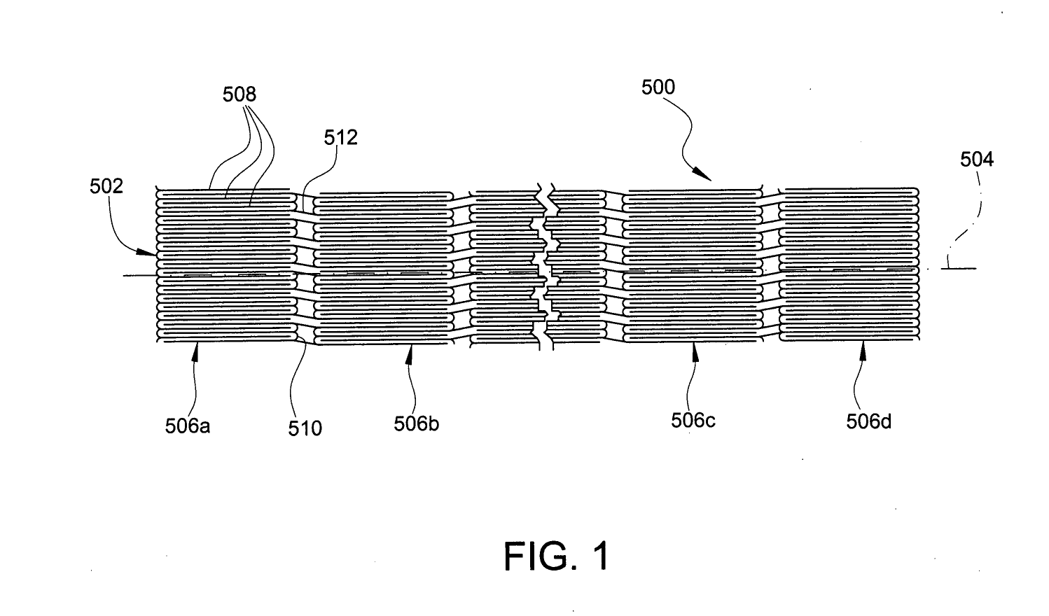

FIG. 1 illustrates a plane development of the endoluminal prosthesis according to an embodiment of the invention, in a collapsed or non expanded state.

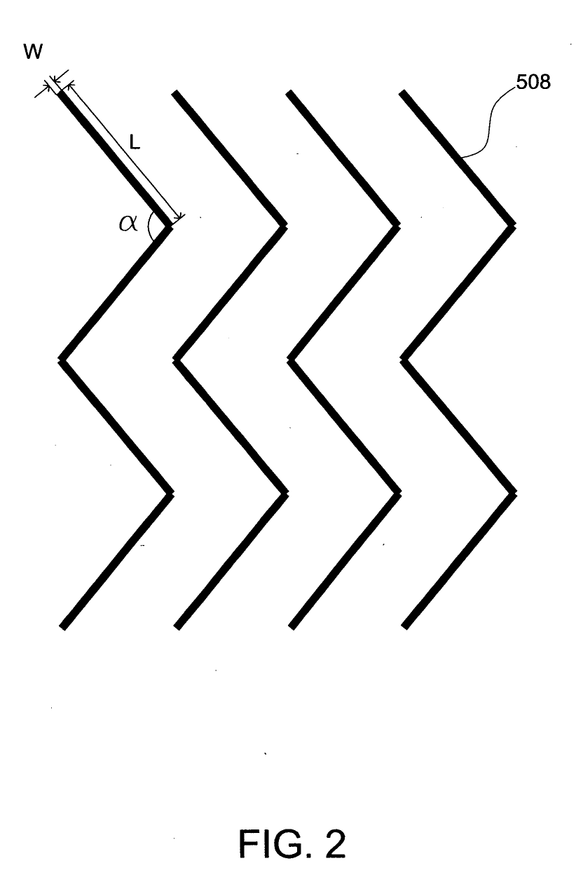

FIG. 2 is a partial, schematic diagrammatic representation of an enlarged detail of the structure of the endoluminal prosthesis according to another embodiment of the invention, in an expanded state.

It is to be understood that none of FIGS. 1 and 2 is a scale drawing.

With reference to the above-mentioned drawings, 500 generally refers to an endoluminal prosthesis, such as a prosthesis of the self-expandable type for ducts or vessels of living bodies, particularly for application in the aorta or cerebral arteries at a site where an aneurysm has occurred.

According to the general embodiment, the endoluminal prosthesis 500 comprises a tubular body 502 suitable to turn from a collapsed state to an expanded or partially expanded state.

By “collapsed state” is meant a condition in which the prosthesis is contracted such as to have a smaller bulk than in an operating condition, e.g. a condition where the tubular body 502 has smaller size or outer diameter than in an operating condition. For example, the prosthesis is arranged in a collapsed state when it is either accommodated or arranged on a transport and delivery device suitable to travel along a duct or vessel to the area to be treated. For example, in the case of a self-expanding prosthesis, this is accommodated in a sheath such as to be maintained in the collapsed condition until expansion has to be effected.

By “expanded or partially expanded state” is meant a condition in which the prosthesis is free from restrictions, or an operating use condition in which the prosthesis is widened upon pressure contact against the inner surface of a duct or vessel wall.

The tubular body 502 develops along a longitudinal axis 504. By “longitudinal axis” is meant for example either a symmetry axis of a cylindrical body or the stretch axial direction of a tubular body.

The tubular body 502 comprises a plurality of closed paths 506a, 506b, 506c, 506d developing along a substantially circumferential direction (FIG. 1). By “closed path” is meant a closed element that develops around a main direction of stretching. The closed paths are serially arranged along the longitudinal axis 504 and each of them is connected to the one or to the ones immediately adjacent.

Each closed paths comprises strut portions, or struts 508 (FIG. 1) having a length and a width.

A schematic diagrammatic representation of the strut length L and strut width W is provided in FIG. 2, which however is not a scale drawing.

According to the general embodiment of the invention, the ratio of the strut length L to the strut width W is of at least 20. A preferred strut length L to strut width W ratio is comprised between 20 and 50, even more preferably between 30 and 40.

A preferred strut length L is comprised within the range of 3 to 5 mm. Even more preferably, the strut length is of about 4 mm.

A preferred strut width W is comprised within the range of 0.1 to 0.2 mm. Even more preferably, the strut width is of about 0.15 mm.

FIG. 2 also illustrates the angle α defined between two consecutive struts of the same closed path when the endoluminal prosthesis is in the expanded state. The angle α is preferably comprised within the range of 90° to 150°, even more preferably between 100° and 120°.

The Applicant has found that the above-mentioned ratio between the strut length L and the strut width W, and optionally the above-mentioned value of the angle α, advantageously provides an endoluminal prosthesis which, when located in its expanded state inside the vessel at the site of an aneurysm, has the following advantageous features and properties: (i) it exerts a low radial force in the radial direction from the vessel wall towards the inside of the blood vessel, so that it easily conforms to the aneurysm and can be easily deformed when compressed from the outside, without exerting an undesirable high pressure thereon; and, at the same time (ii) it offers a high mechanical resistance in the radial direction from the inside of the blood vessel towards the vessel wall, so that the blood flowing within the vessel does not press onto the vessel wall, thereby not causing further deformation of the damaged area.

The above-illustrated features and properties are highly advantageous in that the endoluminal prosthesis of the present invention, when positioned at the site of an aneurysm, is capable of adhering to the vessel wall but, at the same time, does not cause the aneurysm to grow further. This makes it possible for the surgeon to implant the endoluminal prosthesis of the invention, which is not a covered one, at a very early stage of the aneurysm formation, as soon as the presence of the aneurysm is detected e.g. by angiography. After a few months from implantation, the endoluminal prosthesis is incorporated within the vessel wall by the ingrowth of the vessel wall tissue into the prosthesis. Usually the implanted prosthesis is incorporated into the vessel wall after 1-2 years from the implant and is stabilized after 2-3 year from the implant. As a consequence, since the endoluminal prosthesis of the present invention is firmly stabilized within the vessel wall, said prosthesis is capable of acting as a metal mesh and is capable of withstanding the compression force exerted by the blood flow. As a result, further growth of the aneurysm is prevented and the vessel is advantageously protected from rupture thereof. Thus, this kind of endograft is quite similar to a wall vessel scaffolding over which neo-endothelium will constitute an autologous sleeve.

The endoluminal prosthesis of the invention is manufactured to have a predetermined expanded diameter which allows the prosthesis to reach the damaged vessel wall when it is released from the transport and delivery device such as a catheter device. Moreover, since the endoluminal prosthesis of the invention can be easily compressed from the outside towards its longitudinal axis, the pressure exerted by the endoluminal prosthesis onto the vessel wall is low and thus it does not cause expansion of the area which is proximal and distal to the aneurysm.

FIG. 1 shows an embodiment of the endoluminal prosthesis 500 in a collapsed state, in which the closed paths 506a, 506b, 106c, 506d developing along a substantially circumferential direction comprise bend portions, or bends 510 joining subsequent strut portions to form a closed meander path or serpentine.

It is however to be understood that the closed paths 506a, 506b, 106c, 506d may have any shape and may even be devoid of any bend portions.

According to the embodiment illustrated in FIG. 1, each of said paths is connected to the immediately adjacent path(s) by a plurality of bridges (512) substantially extending along the longitudinal axis.

It is however to be understood that the presence and the number of bridges 512, as well as their shape and arrangement, are not essential features of the invention.

FIG. 2 illustrates a schematic enlarged detail of the structure of the endoluminal prosthesis 500 in which the closed paths are in the expanded state and the bridges 512 are not shown.

The endoluminal prosthesis of the invention is made as one piece. For example, the tubular body is obtained by cutting a tubular element, preferably by laser cutting. Advantageously, said body is made of nitinol. Other suitable materials are superelastic materials.

1. An endoluminal prosthesis (500) comprising:
a tubular body (502) suitable to turn from a collapsed state to an expanded or partially expanded state,
said tubular body (502) developing along a longitudinal axis (504),
said tubular body comprising a plurality of closed paths (506a, 506b, 106c, 506d) developing along a substantially circumferential direction,
said paths being serially arranged along the longitudinal axis (504),
each of said paths being connected to the one(s) immediately adjacent,
each of said paths comprising strut portions, or struts (508),
each strut portion being characterized in that the ratio of the strut length (L) to the strut width (W) is of at least 20.

2. The endoluminal prosthesis according to claim 1, wherein the ratio of the strut length (L) to the strut width (W) is comprised within the range of 20 to 50.

3. The endoluminal prosthesis according to claim 2, wherein the ratio of the strut length (L) to the strut width (W) is comprised within the range of 30 to 40.

4. The endoluminal prosthesis according to any of claims 1 to 3, wherein the strut length (L) is comprised within the range of 3 to 5 mm.

5. The endoluminal prosthesis according to claim 4, wherein the strut length (L) is of about 4 mm.

6. The endoluminal prosthesis according to any of claims 1 to 5, wherein the strut width (W) is comprised within the range of 0.1 to 0.2 mm.

7. The endoluminal prosthesis according to claim 6, wherein the strut width (W) is of about 0.15 mm.

8. The endoluminal prosthesis according to any of claims 1 to 7, wherein the angle (α) defined between two consecutive struts of the same closed path when the endoluminal prosthesis is in the expanded state is comprised within the range of 90° to 150°.

9. The endoluminal prosthesis according to claim 8, wherein the angle (α) is comprised within the range of 100° to 120°.

10. The endoluminal prosthesis according to any of claims 1 to 9, which is made of nitinol.

11. The endoluminal prosthesis according to any of claims 1 to 10, wherein said paths are closed meander paths or serpentines.

12. The endoluminal prosthesis according to any of claims 1 to 11, wherein each of said paths comprises bend portions, or bends (510) joining subsequent strut portions to form said serpentines.

13. The endoluminal prosthesis according to any of claims 1 to 12, wherein each of said paths is connected to the immediately adjacent path(s) by at least one bridge (512) substantially extending along the longitudinal axis.
